(19) United States
(12) Patent Application Publication
(10) Pub. No.: US 2023/0151302 A1
(43) Pub. Date: May 18, 2023

(54) Cationic surfactants, in particular ethyl lauroyl arginate LAE, for treating or preventing infections and contaminations with Coronavirus

(71) Applicant: Laboratorios Miret, S.A., Terrassa, Barcelona (ES)

(72) Inventors: Jordi Miret Carceller, Barcelona (ES); Alexis Casanova Morisco, Cardedeu (ES); Carles Lozano Pérez, Montgat (ES); Roger Segret Pons, Barcelona (ES)

(21) Appl. No.: 17/918,171

(22) PCT Filed: Dec. 6, 2021
(86) PCT No.: PCT/EP2021/084335

§ 371 Date: Oct. 11, 2022

(30) Foreign Application Priority Data
Dec. 8, 2020 (WO) 20212380.8

(51) Int. Cl. C11D 1/52; C11D 3/48

(52) U.S. Cl. CPC C11D 1/528; C11D 3/48

(57) ABSTRACT

Disclosed is a method of treatment of surfaces to prevent or treat their contamination with Coronavirus, characterized in that a cationic surfactant of the following formula (1)

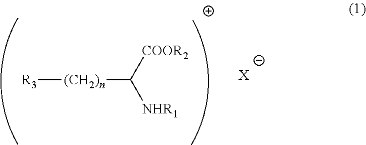

where:
X⁻ is Br⁻, Cl⁻, or HSO4⁻, a counter ion derived from an organic or inorganic acids, or an anion on the basis of a phenolic compound;
R₁ is a linear alkyl chain from a saturated fatty acid or hydroxy acid from 8 to 14 atoms of carbon bonded to the α-amino acid group through an amidic bond,
R₂ is a linear or branched alkyl chain from 1 to 18 carbon atoms or an aromatic group,
R₃ is

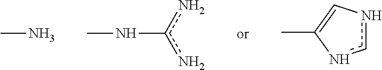

and n can be from 0 to 4,
is applied to the surface.

Particularly preferred is the use of the cationic surfactant of formula (2) designated as LAE®, which is the ethyl ester of the lauramide of the arginine monohydrochloride.

The present invention relates to the use of cationic surfactants of formula (1), in particular ethyl lauroyl arginate LAE® of formula (2), for preventing or treating infections with Coronavirus, and to compositions used in a method for treating or preventing such infections.

### BACKGROUND

Cationic surfactants of the following formula (1) are well-known disinfectants and preservatives.

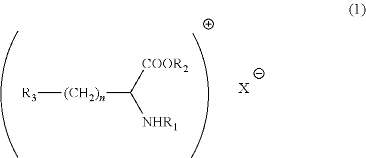

where:
X⁻ is Br⁻, Cl⁻, or HSO4⁻, a counter ion derived from an organic or inorganic acids, or an anion on the basis of a phenolic compound;
R₁ is a linear alkyl chain from a saturated fatty acid or hydroxy acid from 8 to 14 atoms of carbon bonded to the α-amino acid group through an amidic bond,
R₂ is a linear or branched alkyl chain from 1 to 18 carbon atoms or an aromatic group,

### R₃ is

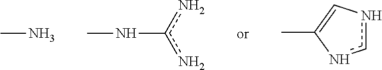

and n can be from 0 to 4.

A preferable well-known substance of formula (1) used for the protection against microorganisms is a cationic surfactant derived from lauric acid and arginine, in particular, the ethyl ester of the lauramide of the arginine monohydrochloride, hereafter named LAE® of formula (2). The compound is also known as “L-arginine, N^{α}-lauroyl-ethyl ester monohydrochloride”, “ethyl-Nα-lauroyl-L-arginate HCl” or “lauric arginate”. The CAS-No. is 60372-77-2. The chemical structure is shown in the following formula (2).

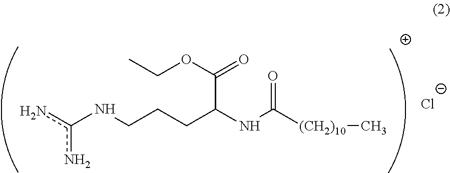

The compounds of formula (1), in particular LAE® of formula (2), have a known activity against some specific virus, namely Influenza virus, in particular Influenza A virus causing avian influenza, Parainfluenza virus, which belong to the subfamilies Orthomyxoviridae and Paramyxoviridae, respectively, Vaccinia virus, Herpes simplex virus and Bovine Parainfluenza virus (WO 2008/014824), and a well-known activity against different microorganisms, such as bacteria, fungi, and yeasts.

WO 2008/095534 discloses a taste-masking composition comprising the cationic surfactant of formula (1), in particular LAE®, and mentions an antiviral activity of LAE® against Vaccinia, Herpes simplex and Bovine Parainfluenza viruses.

The use of the cationic surfactants of formula (1), in particular LAE®, as a preservative against bacteria, fungi and yeasts is known in food and feed preparations.

The compounds of formula (1), in particular LAE®, are well-known to be safe for animals and humans. The metabolism of the above cationic surfactant of formula (1), in particular of LAE®, in rats has been studied, and these studies have shown a fast absorption and metabolization into naturally-occurring amino acids and the fatty acid lauric acid, which are eventually excreted as carbon dioxide and urea. Toxicological studies have demonstrated that LAE® is completely harmless to animals and humans.

It is known that the cationic surfactants of the invention and in particular LAE® and related products are particularly suitable to be used as a preservative in cosmetic preparations (WO 03/013453) and oral care formulations (WO 2009/101115).

The general preparation of the compound of formula (1) is described in ES-A-5124643 and WO 96/21642, WO 01/94292 and WO 03/064669.

LAE® is produced by Laboratorios Miret S.A (LAMIRSA) and is marketed by VEDEQSA under the trademarks Mirenat® and Aminat®.

It is known that LAE® can be used in sanitizers and cosmetic formulations and preparations that are applied in the epidermis, the capillary system, lips, nails, external genital organs, or in the teeth and mouth cavity mucous, in order to clean, perfume, or modify its aspect and/or protect the physical fitness.

For the cationic surfactants of formula (1), in particular LAE®, the antibacterial activity and the biological activity against other microorganisms such as fungi and yeasts is well documented. An activity of the cationic preservatives against specific viruses, i.e. Herpes simplex virus type 1, Vaccinia virus (Orthopoxvirus) and Bovine Parainfluenza 3 virus (ATCC VR-281) has been disclosed in WO 2008/014824. LAE® in water at a concentration of 200 ppm was tested, respectively, and LAE® proved to be effective after 5 and 60 minutes. No remaining virus was detected after 5 and 60 minutes, respectively. The number of virus particles was determined by a quantitative assay determining the TCID₅₀. All viruses tested in WO 2008/014824 are enveloped viruses.

Safe and effective products for the treatment of viral infections are urgently and constantly needed. World-wide outbreaks of virus infections illustrate the complexity of effective treatments and prevention of spreading of the disease. Recently the outbreak of Coronavirus SARS COVID-19 led to an urgent search of the optimum tools to limit the spreading and disease.

Severe Acute Respiratory Syndrome Coronavirus 2 (SARS-CoV-2), previously known as “2019 novel Coronavirus”, is a known as simply the Coronavirus. It has also been called Human Coronavirus 2019 (HCoV-19 or hCoV-19). The World Health Organization declared the outbreak of a Public Health Emergency of International Concern on 30 Jan. 2020 because of HCoV-19, and a pandemic on 11 Mar. 2020. The disease caused by said virus has been designated as SARS COVID-19 or abbreviated COVID-19.

The causative agent involved in the current outbreaks of coronavirus disease 2019 (COVID-19), SARS-CoV-2 (genus: Betacoronavirus), belongs to the family of Coronaviridae, a large family of enveloped, positive-sense single-stranded RNA viruses.

Research into the natural reservoir of the virus strain that caused the 2002-2004 SARS outbreak has resulted in the discovery of many SARS-like bat coronaviruses, most originating in the Rhinolophus genus of horseshoe bats. Phylogenetic analysis indicates that samples taken from Rhinolophus sinicus show a resemblance of 80% to SARS-CoV-2. Phylogenetic analysis also indicates that a virus from Rhinolophus affinis, collected in Yunnan province and designated RaTG13, has a 96% resemblance to SARS-CoV-2. Bats are considered the most likely natural reservoir of SARS-CoV-2, but differences between the bat coronavirus and SARS-CoV-2 suggest that humans were infected via an intermediate host. Although studies have suggested some likely candidates, the number and identities of intermediate hosts remain uncertain. Nearly half of the strain's genome has a phylogenetic lineage distinct from known relatives.

Human Coronavirus 229E (HCoV-229E) virus, a species of Coronavirus which infects humans and bats, and Severe Acute Respiratory Syndrome Coronavirus 2 (SARS-CoV-2) display the same sensibility against external influences. It is belief in the art, that Human Coronavirus 229E (HCoV-229E) virus is a good model for the determination of the biological activity of the compounds against Severe Acute Respiratory Syndrome Coronavirus 2 (SARS-CoV-2) (see ECDC Technical Report “Interim guidance for environmental cleaning in non-healthcare faciliteis exposed to SARS-CoV-2”, 18 Feb. 2020, European Centre for Disease Prevention and Control, Stockholm, 2020 and for example M. Saknimit et al., “Virucidal Efficacy of Physico-chemical Treatments against Coronaviruses and Parvoviruses of Laboratory Animals”, Exp. An. 1988, 37(3), 341-345, in particular p. 344 right col.); R. L. Hulkower et al., “Inactivation of surrogate coronaviruses on hard surfaces by health care germicides”, American Journal of Infection Control 2011, 39(5), 401-407; S. A. Sattar et al., “Chemical disinfection of non-porous inanimate surfaces experimentally contaminated with four human pathogenic viruses”, Epidem. Inf. 1989, 102(3), 493-505), J. Signer et al., “in vitro virucidal activity of Echinaforce®, an Echinacea purpurea preparation, against coronaviruses, including common cold coronavirus 229E and SARS-CoV-2”, Virology Journal 2020, 17:136; Y. Xian et al., “Bioactive natural compounds against human coronaviruses: a review and perspective”, Acta Pharmaceutica Sinica B 2020, 10(7), 1163-1174.

Signer et al. (see above), discloses pon p.a 2/3 that, since HCoV-229E is a typical representative of coronavirus strain causing a seasonal common cold, they used this viruses strain to investigate the general activity of Echinaforce® against coronavirus, thereby closing the gap on other antiviral effects of Echinacea purpurea on typical common cold cold viruses. The document further discloses on p.7 of 11, line 17-22 that the virucidal activity of Echinaforce® against these viruses has been observed. The observed effects against MERS_CoV ans SARS-CoV-1 were comparable with effects observed for HCoV-229E, with complete inactivation after treatment with 50 μg/ml.

V. B. O'Donnell et al., “Potential Role of Oral Rinses Targeting the Viral Lipid Envelope in SARS-CoV-2 Infection”, Function, 2020, 1(1), 1-11 discloses that not all enveloped viruses are the same, and herpes, influenza, and measles viruses are considered more unstable than human coronaviruses, which may persist for up to 5 days on inanimate surfaces. 19,36,96 Thus, research needs to focus on coronaviruses in particular. The exact composition of the SARS-CoV-2 lipidome needs to be determined using lipidomics mass spectrometry. Research should determine the impact of ethanol or other agents on the infective activity of the spike protein itself, in vitro, and in vivo. A useful virus to test in vitro would be the human respiratory coronavirus 229E which is used extensively as a surrogate for human coronaviruses but only requires Category (Cat2) procedures, and its replication and propagation conditions are well established already. This would be a good representative for pathogenic coronaviruses, prior to narrowing down to SARS-CoV-2 which requires Cat3 biosecurity.

It is a remarkable fact that the list N of the EPA (United States Environmental Protection Agency) does not mention LAE® as effective against Coronavirus for use on surfaces. The list N is described as follows: “EPA expects all products on List N to kill the coronavirus SARS-CoV-2 (COVID-19) when used according to the label directions.” In particular, list N of the EPA mentions 1,2-hexanediol, ammonium bicarbonate, ammonium carbonate, chlorine dioxide, citric acid, dodecyl benzensulfonic acid, ethanol, glutaraldehyde, glycolic acid, hydrochloric acid, hydrogen chloride, hydrogen peroxide, hypochlorous acid, iodine, isopropanol, L-lactic acid, octanoic acid, peroxyacetic acid, peroxyoctanoic acid, phenolic, potassium peroxymonosulfate, quaternary ammonium such as Maquat® types and Penetone® types, silver, silver ion, sodium carbonate, sodium carbonate peroxyhydrate, sodium chloride, sodium dichloroisocyanate, sodium dichloroisocyanate dihydrate, sodium hypochlorite, tetraacetyl ethylenediamine, thymol, and triethylene glycol. The quaternary ammoniums mentioned in said list N are benzalkonium chloride, alkyl dimethyl ethylbenzyl ammonium chloride, octyl decyl dimethyl ammonium chloride, dioctyl dimethyl ammonium chloride, didecyl dimethyl ammonium chloride, dimethyl ethylbenzyl ammonium chloride, dimethyl ethylbenzyl ammonium chloride and some similar quaternary ammonium salts.

The corresponding Health Canada's list of hard-surface disinfectants for use against SARS-CoV-2 mentions, in addition, chlorhexidine gluconate and benzethonium chloride. LAE® is not mentioned in the Health Canada's list of disinfectants against Coronavirus, either.

The antiviral disinfectant recommended by the WHO consists of 83.3 vol % ethanol (96%), 4.17 vol. % hydrogen peroxide (3%), 1.45 vol. % glycerol (98%) and balance sterile water.

S.A. Sattar et al. (see above) have found that quaternary ammonium compounds, which are frequently used for general hard surface disinfection, did not even inactivate the enveloped viruses HPIV-3 and human Coronavirus 229E.

E. Statkute et al., “Brief Report: The Virucidal Efficacy of Oral Rinse Components Against SARS-CoV-2 in vitro”, Abstract, bioRxiv, 18 Nov. 2020, p. 1-10, XP055799087, DO1:10.1101/2020.11.13.381079 reports an in vitro test of various commercially available mouthwash formulations for inhibiting SARS-CoV-2 and/or HCoV-229E infection. One of the liquid preparations tested in this study is “Listerine Advanced Gum Treatment” which, according to this report, contains 0.147% w/w ethyl lauroyl arginate HCl and 23% v/v ethanol. Listerine Advanced (23% ethanol) with ethyl lauroyl arginate (LAE), according to this document, eradicated the virus completely giving >5-log 10 reduction in viral titres. The study describes that ethanol alone at <23% had no effect on virus infectivity and that the inclusion of essential oils (Listerine Cool Mint) or LAE (Listerine Advanced) appears to be required for optimal efficacy, and that chlorhexidine was relatively inactive (<2 log fold reduction).

The website of the manufacturer of Listerine® mouthwash shows several Listerine® Advanced products, i.e. “Listerine® Advanced White Milder Taste”, “Listerine® Advanced Defence Gum Treatment” and “Listerine® Advanced Defence Sensitive”. Of these different mouthwashes, the product “Listerine® Advanced Defence Gum Treatment” is the only one containing LAE®. According to the manufacturer's website “https://www.listerine.co.uk/products/healthy-gums/listerine-advanced-defenced-gum-treatment#ingredients”, the product “Listerine®Advanced Defence Gum Treatment” contains ethyl lauroyl arginate HCl (LAE) in a concentration of 0.147% w/w and alcohol in an undisclosed amount, besides purified water, sorbitol, glycerin, Poloxamer 407, aroma, benzoic acid, sucralose and sodium benzoate.

T. L Meister et al., “Brief Report: Virucidal Efficacy of Different Oral Rinses Against Severe Acute Respiratory Syndrome Coronavirus 2”, JID 2020, 1289-1292 describes result of a study investigating the effect of commercially available mouthwash preparations for inactivating properties for SARS-CoV2 under in vitro conditions mimicking nasopharyngeal secretions. Several commercially available mouthwashs significantly inactivated SARS-CoV-2 within 30 seconds exposure time. One of the preparations was Listerine® Cool Mint, the active ingredients thereof being ethanol and essential oils. It reduced viral infectivity to up to 3 orders of magnitude to background levels and was similarly effective as iso-Betadine® mouthwash (Active ingredient: povidone-iodine) and Dequonal® mouthwash (Active ingredient: dequalinium chloride, benazlkonium chloride).

WO 2011/119517 A2 discloses environmentally beneficial compositions that include a cationic surfactant and certain antimicrobial agents or preservatives. Useful cationic surfactants disclosed include LAE®. The document states in a general manner that LAE® is active against Coronavirus including SARS-CoV. LAE, according to this document, is used in combination with other antimicrobials such as quaternary ammonium compounds, phenolic compounds or 1,2-methyl-1,2-thiazol-one or with preservatives such als alcohols and alkanediols. The tests reported for LAE® are against several bacteria and some specific viruses, i.e. Rotavirus and Influenza A virus.

### OBJECT OF THE INVENTION

The object of the invention is to provide a new use of the cationic surfactant of formula (1), in particular LAE®, for treating or preventing infections with Coronavirus types, and a composition for said use comprising the cationic surfactant of formula (1), in particular LAE®.

Surprisingly the inventors have found that the cationic surfactant of formula (1), in particular LAE®, acts as a virucide against Coronavirus at a very low concentration in a very short contact time.

Therefore, it can be expected to be a very effective disinfectant/sanitizer to erase Coronavirus from surfaces of any type.

It can further be expected that it will be effective as virucide in cosmetic, body care and/or oral care formulations and preparations. Coronavirus may be present in such preparations due to deposition on the epidermis, the capillary system, lips, nails, external genital organs, or in the teeth and mouth cavity mucous by the regular life. The cationic surfactant may prevent the spreading of Coronavirus when present in cosmetic, body care and/or and/or oral care formulations.

It is further expected that the cationic surfactant of the invention, in particular LAE®, will be a viricidally active medicament for preventing or treating Coronavirus infections.

Surprisingly, the antiviral activity of the cationic surfactant of formula (1), in particular LAE®, could be observed against a Coronavirus type, and a very short contact time of 1 minute was sufficient for achieving maximum effects, specifically a reduction bigger than 4 logo. This may be considered as a particular surprising effect of the present invention, i.e. achieving the strong effect against the viruses after such a short time.

It was shown in confidential studies carried out by Instituto Valenciano de Microbiologia, Bétera (Valencia), Spain conducted for the applicant, the results of which are given in the Reference Examples of this application, that LAE® is not effective against Poliovirus (which is a non-enveloped RNA virus) at the maximum dose tested of 1000 ppm, but that it is somehow effective against Adenovirus (non-enveloped DNA virus) and murine Norovirus (non-enveloped RNA virus) in concentrations of 1000 ppm and 500 pm, whereas it is not effective against these two viruses in a lower concentration of 50 ppm, all in a contact time of 30 minutes, respectively.

The viruses specifically disclosed in WO 2008/014824 and WO 2008/095534, where LAE® was effective after 5 minutes and 60 minutes at a concentration of 200 ppm, are all enveloped virus.

However, the activity of the cationic surfactant of formula (1), in particular LAE®, against Coronavirus, which is an enveloped virus, as shown in a confidential study carried out by the same Institute for the applicant, the results of which are presented as Example 1 below, is surprisingly high since it is effective in a concentration as low as 50 ppm, and already after 1 minute. This very high activity could not have been expected by the skilled person.

The present invention provides the aspects shown in the appended claims:

The organic acids which may be the source of the counter ion X⁻ in the compound of formula (1) can be citric acid, lactic acid, acetic acid, fumaric acid, maleic acid, malic acid, gluconic acid, propionic acid, sorbic acid, benzoic acid, carbonic acid, glutamic acid, tartaric acid, succinic acid or other amino acids, lauric acid and fatty acids such as oleic acid and linoleic acid, whereas the inorganic acids can be phosphoric acid, nitric acid and thiocyanic acid.

The phenolic compound which may be the basis of the anion X⁻ of formula (1) is for instance butylated hydroxyanisole (BHA) and the related butylated hydroxytoluene, tertiary butyl hydroquinone and parabens such as methylparaben, ethylparaben, propylparaben and butylparaben.

The most preferred compound of the above class of compounds of formula (1) is LAE® of formula (2).

The present invention, in particular, relates to the use of the cationic surfactant of formula (1), in particular LAE®, for treating any kind of objects and/or surfaces which may be affected by a Coronavirus contamination. Such products may for instance be warm dishes or warm liquids. The products to be treated may be any kind of equipment which is used in the handling of animals which are infected with virus. In an even broader sense the products to be treated can be the facilities where animals are kept, or parts of the natural environment such as the ground surface or water reservoirs. The products to be treated can further be facilities where human beings are treated such as hospitals or medical practice or any environment where a spreading of a virus disease could occur, such as office buildings, public transport buildings and vehicles, sports locations, houses, cinemas and the like.

The present invention furthermore relates to the administration of the cationic surfactant of formula (1), in particular LAE®, to animals or human beings directly, for prophylactic or therapeutic treatment of Coronavirus diseases.

### EXPLANATION OF THE FIGURES

FIGS. 1 to 4 are graphic representations of the results of Example 1 and Reference Examples 1 to 3.

### DETAILED DESCRIPTION OF THE INVENTION

An infection, according to the invention, is the invasion of an organism's body tissues by disease-causing agents, their multiplication, and the reaction of host tissues to the infectious agents and the toxins they produce. An infectious disease, also known as a transmissible disease or communicable disease, is an illness resulting from an infection.

A contagion occurs in a contagious disease. A contagious disease is a subset category of transmissible diseases, which are transmitted to other persons, either by physical contact with the person suffering the disease, or by casual contact with their secretions or objects touched by them or airborne route among other routes.

Contamination, in the invention, is the presence of a constituent, impurity, or some other undesirable element that spoils, corrupts, infects, makes unfit, or makes inferior a material, physical body, natural environment, workplace, etc. In particular, contamination with Coronavirus is implied in the invention.

The cationic surfactant of formula (1), in particular LAE® of formula (2), may be applied as a solution. This is the easy and suitable manner of treating the surface of the ground, vehicles, medical devices, animals and people. For other applications it may be more suitable to apply the cationic surfactant as a solid, which may be equally effective.

It is preferred to dissolve the compound directly before use in the following preferred solvents of cosmetic or food grade: water, ethanol, propylene glycol, glycerol, isopropyl alcohol, other glycols, mixtures of glycols and mixtures of glycols and water. If the treatment shall be performed at a specific pH value the use of a corresponding buffer solution may be recommendable. On the other hand, the cationic surfactant, in particular LAE®, can be used as a solid. Surfaces which shall be protected by solid preparations are for instance the surfaces of food products, cosmetics and/or oral care formulations and preparations.

A typical concentration of the cationic surfactant, in particular LAE®, in food products is between 1 ppm and 10,000 ppm. A preferred concentration is in the range of 1 to 1,000 ppm, a more preferred range between 10 and 200 ppm, an even more preferred range between 10 and 100 ppm. Although the preferred ranges are in a low concentration range, the use in the higher concentrations is regularly observed and is according to the invention.

A typical concentration of the preservatives of formula (1), in particular LAE®, in cosmetic preparations is between 1 ppm and 15,000 ppm. A preferred concentration is in the range of 200 to 10,000 ppm, a more preferred range between 500 and 10,000 ppm, an even more preferred range between 800 and 8,000 ppm. The use in higher or lower concentrations is often observed and is according to the invention.

The treatment of products in order to avoid any kind of virus infection or contamination might involve the presence of a concentration of the cationic surfactant of formula (1), in particular LAE®, of around 2 to 20,000 ppm of the product to be protected, preferably a concentration of 100 to 10,000 ppm, and more preferably 200 to 2,000 ppm. This is a similar concentration as has been described for achieving the microbiocidal action. Products to be treated with the above-indicated range of concentrations of the cationic surfactants are for instance food products or cosmetics.

The treatment of surfaces which are contaminated with viruses, such as the surface of food preparations, the surface of food contact materials, the surface of cosmetics, ground surface, the surface of any kind of vehicles, and the surface of any equipment used in the handling of persons or animals infected with virus, textiles, or medical equipment, requires the presence of the cationic surfactant of formula (1), in particular LAE®, at a concentration of a level which is sufficient to achieve the wanted antiviral action at such surfaces. Such level of concentration would be expected in the range of 10 ppm to 20,000 ppm, more preferred 50 ppm to 10,000 ppm and even more preferred 100 to 5,000 ppm. These concentrations are given in terms of the concentration of a solution containing the cationic surfactant which is applied to the surfaces to be treated.

For the inventive use as a sanitizing viricidal liquid or gel for skin disinfection, the concentration of the cationic surfactant, in particular LAE®, is usually 2 to 20,000 ppm of the product to be protected, preferably a concentration of 50 to 10,000 ppm, and more preferably 100 to 2,000 ppm.

If surfaces are treated with a solid preparation of the cationic surfactant, in particular LAE®, the amount which is applied shall be such that the amount of cationic surfactant, in particular LAE® shall be in the range of 0.01 to 1000 mg/dm², preferably 0.01 to 100 mg/dm², more preferably 0.5 to 100 mg/dm², even more preferably an amount of 0.5 to 50 mg/dm², and most preferably an amount of 1 to 10 mg/dm².

The treatment of liquid preparations such as drinking fluids or natural sources of water such as lakes or ponds requires the presence of the cationic surfactant of formula (1) in particular LAE®, at a concentration of a level which is sufficient to achieve the wanted antiviral action in the drinking fluid or water. Such level of concentration would be expected in the range of 0.2 to 20,000 ppm, more preferred 2 to 15,000 ppm, even more preferred 50 to 10,000 ppm and most preferred 100 to 2,000 ppm, cationic surfactant. These concentrations are provided in terms of the concentration of the cationic surfactant in the liquid or the water to be treated.

In liquid formulations, such as a mouthwash, the concentration of LAE® based on the total formulation is preferably 1,200 ppm or less, more preferably 1,000 ppm or less. The lower limit of the LAE® concentration in liquid formulations such as moutwash is preferably 50 ppm or more, more preferably 100 ppm or more and most preferably 500 ppm or more. The preferred ranges of concentration of LAE® in liquid preparations such as mouthwash is 100 to 1,200 ppm, preferable 500 to 1,000 ppm.

The treatment of animals or humans implies the administration of the cationic surfactant in a manner which is suitable for absorption of the cationic surfactant, in particular used according to the invention. The compound of formula (1), in particular LAE®, can be administered orally, parenterally (including intraperitoneal, subcutaneous and intramuscular injections) or externally (topically, such as rectal, transdermal, by instillation and transnasal or as mouthwash). The preparation to be administered may have the form of a conventional pharmaceutical preparation such as capsules, microcapsules, tablets, enteric coated agents, granules, powder, pills, ointments, suppositories, suspensions, syrups, emulsions, liquids, sprays, inhalants, eye drops and nose drops.

The above-mentioned pharmaceutical and cosmetic preparations can be produced according to conventional methods using various organic or inorganic carriers conventionally used for the pharmaceutical formulation of preparations, such as excipients (such as sucrose, starch, mannite, sorbite, lactose, glucose, cellulose, talc, calcium phosphate, calcium carbonate), binders (such as cellulose, methyl cellulose, hydroxymethyl cellulose, polyvinylpyrrolidone, gelatine, Arabic gum, polyethylene glycol, sucrose, starch), disintegrants (such as starch, carboxymethyl cellulose, hydroxypropyl starch, sodium hydrogen carbonate, calcium phosphate, calcium citrate), lubricants (such as magnesium stearate, Aerosil®, talc, sodium lauryl sulphate), corrigents (such as citric acid, menthol, glycine, orange powder), preservatives (such as sodium benzoate, sodium bisulfite, methylparaben, propylparaben, benzalkonium chloride), stabilizers (such as citric acid, sodium citrate, acetic acid), suspending agents (such as methyl cellulose, polyvinylpyrrolidone, aluminium stearate), dispersing agents (such as hydroxypropyl methyl cellulose), diluents (such as water, ethanol, propylene glycol, glycerol, isopropyl alcohol, other glycols, mixtures of glycols and mixtures of glycols and water), base waxes (such as cacao butter, white petrolatum, polyethylene glycol) and other suitable ones.

The compositions of the invention preferably comprise a medium which is compatible with the skin, the mucous membranes, hair and food preparation surfaces. Besides the components mentioned before, compositions may comprise mineral oil, animal oil, vegetable oil, synthesis and silicon oils, waxes, fatty acids, fatty acids salts, organic solvents, surface active ingredients, solubilizers and ionic and non-ionic emulsifiers or surfactants (e.g. Polysorbate 20, Polysorbate 40, Polysorbate 60, Polysorbate 61, Polysorbate 65, Polysorbate 65, Polysorbate 80, Polysorbate 81, Polysorbate 85), thickening agents and gelling agents, such as carboxyvinylic polymers (e.g. Carbomer), acrylic copolymers, (e.g. acrylates and alkylacrylates), polyacrylamides, polysaccharides, natural gums (e.g. Xanthan gum), lipophilic agents such as modified clays (e.g. bentonite), fatty acid metallic salts, hydrophobic silica and polyethylene, perfume and essential oils, softening agents, antioxidants, sequestering (chelating) agents (e.g. tetrasodium EDTA and tetrahydroxypropyl ethylenediamine), opacifiers, filters, colouring compounds which may be either hydrosoluble or liposoluble, and natural or synthetized pigments, and hydrophilic or lipophilic active ingredients.

According to the invention the compositions can be in different forms suitable for topic, or surface application:
a) monophasic systems
b) biphasic systems
c) combinations of the other systems that form multiphasic systems, suspensions or microemulsions.

The compositions previously mentioned can be used in different forms, such a foam, spray, or aerosol composition and can contain a propulsion agent under pressure.

Thus, the compositions of the invention can have the aspect of a cream, a lotion, a milk, an emulsion, a gel or an oil for the skin, a beauty mask, a salt, a hair conditioner, a gel, including sanitizer gel, a foam/spray, an oil for a bath and shower, a liquid soap, or a make-up and make-up remover for the face and the eyes, and any other aspect known in the art.

The dose of cationic surfactant of formula (1), in particular LAE®, according to the prophylactic or therapeutic use of the present invention shall be determined by the dose required for achieving the desired prophylactic or therapeutic effect. A usual dose shall be 0.1 mg/kg to 10 mg/kg for oral or parenteral administration. A usual dose in humans may be a unit dose of 0.1 to 1000 mg per individual, more preferable 0.5 to 500 mg per individual. This dose may be administered 1 to 4 times per day, depending on the severity of the symptoms. A usual dose in animals may be 0.1 to 100 mg per dose, preferred 0.5 to 50 mg per dose.

The compositions of the invention can be used for hygienic handrub, hygienic handwash, instrument disinfection by immersion, surface disinfection by wiping, spraying, flooding or other means, and for textile disinfection.

They can be applied where disinfection is medically indicated such as in hospitals, community medical facilities, dental institutions, clinics of schools, kindergartens and nursing homes, in the workplace and in the home. It may also include services such as laundries and kitchens supplying products directly for the patents.

Since the cationic surfactant of formula (1), in particular LAE®; has been shown to be surprisingly effective against Coronavirus 229E, and since this virus type is considered to be a good model for Severe Acute Respiratory Syndrome Coronavirus 2 known as SARS-CoV-2, Severe Acute Respiratory Syndrome Coronavirus known as SARS-CoV, Human Coronavirus 229E known as HCoV-229E or Middle East Respiratory Syndrome-related Coronavirus known as MERS-CoV, in particular for SARS-CoV-2 a particular antiviral effect of the cationic surfactant of formula (1), in particular of LAE®, against SARS-CoV-2 is expected.

The effect of the use of the invention is illustrated through the following examples.

The definitions given in the European standard EN 14476:2013+A2:2019(E), the contents of which is incorporated herein by reference, are applied in the present application.

The tests have been carried out exactly according to said standard in the case of Norovirus, Poliovirus and Adenovirus, and according to an adaptation of said standard for Coronavirus (which specifies only Poliovirus type 1, LSc 2ab (Picornavirus), murine Norovirus strain S99 Berlin, Adenovirus type 5 strain Adenoid 75, ATCC-VR-5 and murine Parvovirus minute virus of mice, strain Crawford, ATCC VR-1346). Besides the type of virus, nothing has been changed in the procedures described in EN 14476:2013+A2:2019(E). This adaptation is designated DESIN-6225 by Instituto Valenciano de Microbiologia, Bétera (Valencia), Spain, where the confidential tests have been conducted. This institution is certified as a technically competent according to ISO 17025 and works under good laboratory practices (GLP).

The principle of the test method is as follows:

The method is an endpoint dilution assay. It quantifies the amount of viruses required to kill 50% of infected host cells or to produce a cytopathic effect in 50% of inoculated tissue culture cells. Host cells are plated and serial dilutions of the virus are added. After incubation, the percentage of cell deaths (i.e. infected cells) is manually observed and recorded for each virus dilution, and results are used to calculate a TCID₅₀ result.

In the present invention, specifically, a sample of the test product (i.e. LAE®) is diluted with hard water to prescribed concentrations (1000 ppm, 500 ppm and 50 ppm, respectively) and is added to a test suspension of viruses in a solution of an interfering substance (i.e. 0.3 g/I bovine serum albumin). The mixture is maintained at the temperature and the contact time specified in the following. At the end of the contact time an aliquot is taken; the viricidal action of the test product in this portion is immediately suppressed by dilution of the sample in ice-cold cell maintenance medium. The dilutions are transferred into cell culture units (in the present invention, 12-well microtiter plates) using cell monolayers. Infectivity tests are done by quantal tests. After incubation, the titers of infectivity (virus titers) are calculated according to the Spearman and Kärber method. Reduction of virus infectivity is calculated from differences of logo virus titers before (virus control) and after treatment with the test product.

The medium for cell cultures is Eagle's minimal essential medium (MEM) or equivalent, supplemented with FCS, antibiotics, and other growth factors as needed according to 5.2.2.10 of the Standard EN 14476:2013+A2:2019 (E).

The growth medium is supplemented with 10% FCS (fetal calf serum) and is obtained by adding 10 pbw of FCS to 90 parts by weight of MEM.

The maintenance medium to maintain the cell culture metabolism without stimulation of proliferation is supplemented with 2% FCS and is obtained by adding 2 parts by weight FCS to 98 parts by weight of MEM.

The cell monolayers are >90% confluent before inoculation. Cells for virus titration are used in suspension in quantal tests and are added to the dilutions of the test mixture in such a density as to enable the formation of a monolayer in at least two days in the cell control.

The test virus and their stock cultures are prepared and kept in accordance with EN 12353. The stock virus suspension is multiplied in an appropriate cell line mentioned below that produces high titers of infectious viruses. The cell debris is separated by centrifugation (400 g_{N} for 15 min). This preparation is designated “test virus suspension”. The minimum titer of the virus suspension—determined by a quantal test—ideally 10⁸TCID₅₀/ml. In any case, it is sufficiently high to at least enable a titer reduction of 4 logo to verify the method.

Adenovirus and Poliovirus is multiplied in HeLa cells, or in other cell lines of appropriate sensitivity. In the present application Vero cells, ref. FTVE cells, have been used.

Norovirus is multiplied in RAW 264.7 cells (ATCC TIB-71) or other cell lines of appropriate sensitivity. In the present case Raw 264.7 Public Health England cells have been used.

Coronavirus, in the present invention, is multiplied in MRC-5, ref. FTMR cells.

Adenovirus used in this application is Adenovirus type 5 (ATCC VR-5). Poliovirus used in the invention is Poliovirus type 1 (ATCC VR-192). Murine Norovirus used in this application is cepa S99 Berlin. Coronavirus used in the present invention is Coronavirus 229E (ATCC VR-740).

### Test Procedure

1 ml of interfering substance (bovine serum albumin, BSA) solution (0.3 g/L BSA) is pipetted into a container. 1 ml of virus test suspension having a titer that is sufficiently high to at least enable a titer reduction of 4 log₁₀, ideally at least 10⁸ TCID₅₀/ml is added. 8 ml product test solution of the prescribed concentration is added and mixed. A stopwatch is started at once and the container is placed in a water bath controlled to the specified temperature. The activity of the test product LAE® is determined for the specified contact times. Immediately at the end of the chosen contact time 0.5 ml of the test mixture is pipetted into 4.5 ml ice-cold maintenance medium and put into an ice bath. Within 30 min a series of ten-fold dilutions of this mixture (test mixture+ice bath) is prepared by changing pipette tips after each dilution to avoid carry-over of viruses. After incubation, the virus titre is calculated, and reduction of virus infectivity is determined from differences of logo virus before and after treatment with the test product.

After treatment with the test product the infectivity is tested with one of the procedures specified in 5.5.2 of the Standard EN 14476:2013+A2:2019 (E), i.e. quantal tests (endpoint titration) on cells in suspension on microtiter plates, virus titration on monolayers of cells on microtiter plates, virus titration on monolayers of cells in cell culture tubes or flasks, or plaque assay (for poliovirus). In the present application the quantal test 2) (virus titration on monolayers of cells on microtiter plates) using a cell monolayer has been used for all viruses tested, including Poliovirus.

In the quantal test, the TCID₅₀ assay (tissue culture infectious dose), a procedure to determine the infectious titre of any virus which can cause viral cytopathic effect (CPE) in cell culture, is read after 1 hour of incubation at 37±1° C.

An interference control is carried out as well. The aim of the interference control is to verify that the susceptibility of the cells for the virus infection is not influenced negatively by the treatment with the product test solution. Comparative virus titrations are performed on cells that have or have not been treated with product test solution to check the reduction of the sensitivity to viruses according to 5.5.4.2 of the Standard EN 14476:2013+A2:2019 (E).

A control of efficiency of suppression of product's activity is carried out by dilution in ice-cold medium (MEM+2% FCS) according to 5.5.5.1 of the Standard EN 14476:2013+A2:2019 (E). The difference of titer with the test suspension shall be ≤0.5 log₁₀.

A reference test for virus inactivation is carried out using formaldehyde as a control of the test system according to 5.5.6.1. of the Standard EN 14476:2013+A2:2019 (E). This reference test is carried out with 0.7% (m/v) formaldehyde and a contact time of 60 minutes in parallel with the tested product LAE® for the internal control of the test.

The cytotoxicity of the formaldehyde solution is control of the validity of the test according to 5.5.6.2. of the Standard EN 14476:2013+A2:2019 (E).

The infectivity of the virus suspension shall be controlled according to 5.5.7 of the Standard EN 14476:2013+A2:2019 (E) by replacing the product test solution by hard water.

The TCID₅₀ is the 50% infecting dose (fifty-percent tissue culture infective dose) of a virus suspension, that is, the dilution of the virus suspension that induces a viral cytopathic effect (CPE) in 50% of cell culture units. The TCID₅₀ is calculated from the test results obtained above by the Spearman-Kärber method described in Annex C of the Standard EN 14476:2013+A2:2019 (E). Prerequisite is the use of several dilutions which cover infection of all cell culture units to those in which no virus multiply. The mean and standard deviation are calculated by obtaining several different test results.

Cytotoxicity means a morphological alteration of cells and/or their destruction or their reduced sensitivity to virus multiplication caused by the tested product (LAE® in the present invention).

The viral cytopathic effect (CPE) is the morphological alteration of cells and/or their destruction as a consequence of virus multiplication.

The virus titer is the amount of infectious virus per unit volume present in a cell culture lysate or in a solution.

There are two options to perform the infectivity test, clean or dirty, and the difference is the concentration of this albumin solution (interfering substance).

Clean conditions mean 0.3 g/I bovine albumin (BSA) solution. In the test used in the present invention according to the standard EN 14476:2013+a2:2019 (E) BSA solution is the interfering substance used. Albumin is the most abundant protein in the human plasma, and a kind of contamination which is “usual” in hospitals and operating rooms. As the viricidal products are normally used in hospitals and operating rooms, use of this interfering substance in the viricidal activity test makes sense.

Dirty conditions mean a mixture of bovine albumin solution—high concentration with sheep erythrocytes.

Hard water is used for dilution of tested products and is water (either glass-distilled water, not demineralised water; or water for injection) containing defined amounts of magnesium chloride, calcium chloride and sodium bicarbonate which is prepared as specified in 5.2.2.7 of the Standard EN 14476:2013+a2:2019 (E).

When LAE® is diluted in said hard water a stable mixture is formed. This means that LAE® is soluble in hard water and the tested solution, or dilution of a solid, does not form precipitates or bi-phasic mixtures. If the liquid or solid tested is, for example, not soluble in the medium (hard water), creates a bi-phasic solution (Example: organic solvents not miscible), or precipitates with hard water, the tests could not be conducted. The obtained solution of LAE® is transparent.

### Example 1

### The Activity of LAE® was Investigated Against Coronavirus

LAE® (CAS 60372-77-2) batch no. 026297A with a LAE® content of 88.4% was provided by Laboratorios Miret, S.A., Terrasa (Barcelona), Spain.

The investigated strain of the Coronavirus was human Coronavirus 229 E (ATCC VR-740).

Experimental Conditions:

LAE® was investigated according to the Standard NF EN 14476: 2013+A2: 2019 at a concentration of 50 ppm, 500 ppm and 1000 ppm, respectively. The contact time was 1 min in each test, and the contact temperature was 20° C.±1° C., respectively. The test was carried out in clean conditions using BSA as an interfering substance. The incubation temperature was 35° C.±1° C. The cellular lines were MRC-5, ref. FTMR, aliquots of work 4, passage 15 and 17, aliquot of work 5, passage 9.

The results are displayed in the following Table 1 and FIG. 1.

TABLE 1
Results of the activity of the product LAE ®, batch 026297A,
against Coronavirus 229E (ATCC VR-740), in clean conditions.

| | | | | | | | | Reduction with |
|---|---|---|---|---|---|---|---|---|
| | | | | log₁₀ TCID₅₀ after | | | | 95% confidence |
| | | Interfering | Level of | 0 | 1 | 5 | 15 | interval after |
| Product | Concentration | substance | cytotoxicity | min | min | min | min | 1 minute |
| LAE ® | 1000 ppm | 0.3 g/L BSA | 0.5 | — | 0.50 | — | — | ≥5.74 ± 0.33 |
| | 500 ppm | | 0.5 | — | 0.50 | — | — | ≥5.74 ± 0.33 |
| | 50 ppm | | 0.5 | | 1.66 | — | — | 4.58 ± 0.45 |
| Virus control | NA | 0.3 g/L BSA | NA | 6.32 | 6.24 | — | — | NA |
| Formaldehyde | 0.7% (m:v) | NA | 0.5 | NR | NR | 3.58 | 2.49 | NA |
| Formaldehyde | 0.7% (m:v) | NA | NA | 5.91 | NR | NR | 5.74 | NA |
| virus control | | | | | | | | |

Control of cell susceptibility (logarithmic difference between viral titers using treated and untreated cells) log10^{-0.57}
Control of the efficacy of suppression of product activity (logarithmic difference between virus titers of the control virus and that of the test suspension log10^{-0.24}
NA: not applicable;
NR: not performed
Times recommended for surfaces: maximum 5 or 60 min
Times recommended for instruments: maximum 60 min
Times recommended for hygienic friction hand treatment and hygienic hand washing: between 30 and 120 seconds
Viricidal activity exists when the virus titer shows a reduction ≥4 log₁₀.
PBS: phosphate saline buffer,
BSA: bovine serum albumin

According to this test, the disinfectant product LAE®, in clean conditions, at concentrations of 1000 ppm, 500 ppm and 50 ppm and with 1 minute exposure, has viricidal activity against Coronavirus 229E (ATCC VR-740), with a reduction of ≥5.74±0.33 TCID₅₀ at the concentration of 1000 ppm, with a reduction of ≥5.74±0.33 TCID₅₀ at the 500 ppm concentration, and with a reduction of 4.58±0.45 TCID₅₀ at the 50 ppm concentration.

### Reference Example 1

### The Activity of LAE® was Investigated Against Poliovirus Type 1

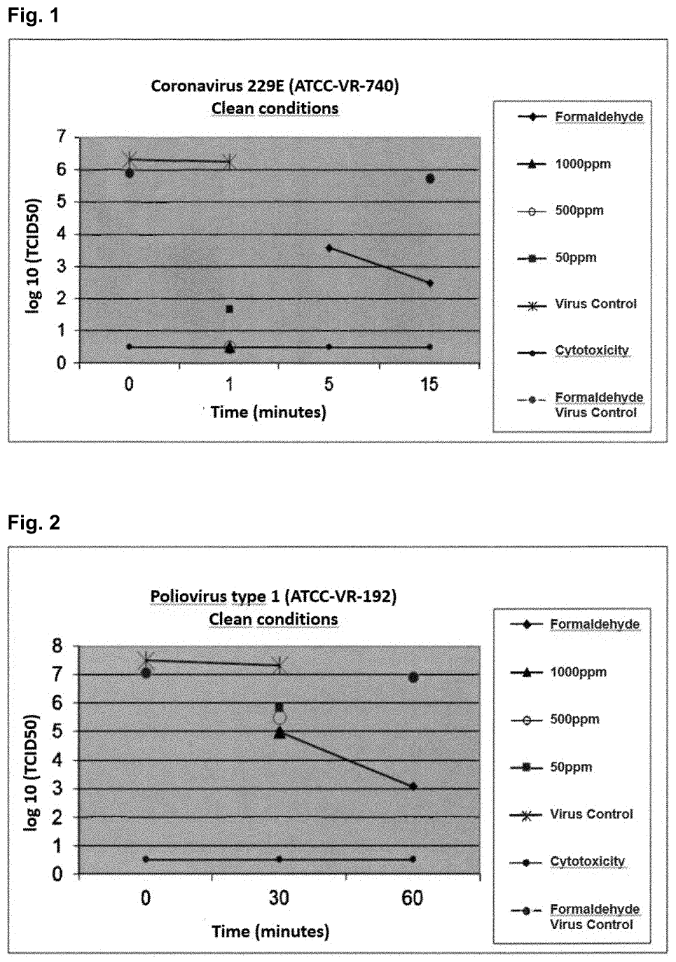

The test was carried out in the same way as in Example 1, but using Poliovirus type 1 (ATCC VR-192). The contact time was 30 minutes, however. The incubation temperature was 37° C.±1° C., and the cellular lines were Vero, ref. FTVE, aliquots of work 5, passages 15 and 17. The results are shown in Table 2 and FIG. 2.

TABLE 2
Results of the activity of the product LAE ®, batch 026297A,
against Poliovirus type 1 (ATCC VR-192), in clean conditions.

| | | | | | | | | Reduction with |
|---|---|---|---|---|---|---|---|---|
| | | | | log₁₀ TCID₅₀ after | | | | 95% confidence |
| | | Interfering | Level of | 0 | 15 | 30 | 60 | interval after |
| Product | Concentration | substance | cytotoxicity | min | min | min | min | 30 minutes |
| LAE ® | 1000 ppm | 0.3 g/L BSA | 0.5 | — | — | 4.99 | — | 2.33 ± 0.53 |
| | 500 ppm | | 0.5 | — | — | 5.50 | — | 1.82 ± 0.52 |
| | 50 ppm | | 0.5 | — | — | 5.83 | — | 1.49 ± 0.46 |
| Virus control | NA | 0.3 g/L BSA | NA | 7.49 | — | 7.32 | — | NA |
| Formaldehyde | 0.7% (m:v) | NA | 0.5 | NR | NR | 5.00 | 3.07 | NA |
| Formaldehyde | 0.7% (m:v) | NA | NA | 7.08 | NR | NR | 6.91 | NA |
| virus control | | | | | | | | |

Control of cell susceptibility (logarithmic difference between viral titers using treated and untreated cells) log10^{-0.42}
Control of the efficacy of suppression of product activity (logarithmic difference between virus titers of the control virus and that of the test suspension) log10^{-0.41}
NA: not applicable;
NR: not performed
Times recommended for surfaces: maximum 5 or 60 min
Times recommended for instruments: maximum 60 min
Times recommended for hygienic friction hand treatment and hygienic hand washing: between 30 and 120 seconds
Viricidal activity exists when the virus titer shows a reduction ≥4 log₁₀.
PBS: phosphate saline buffer,
BSA: bovine serum albumin

Consequently, the disinfectant product LAE®, in clean conditions, at concentrations of 1000 ppm, 500 ppm and 50 ppm and with 30 minutes exposure, has no viricidal activity against Poliovirus type 1, with a reduction of 2.33±0.53 TCID₅₀ at the concentration of 1000 ppm, with a reduction of 1.82±0.52 TCID₅₀ at the 500 ppm concentration, and with a reduction of 1.49±0.46 TCID₅₀ at the 50 ppm concentration.

### Reference Example 2

### The Activity of LAE® was Investigated Against Adenovirus Type 5

The test was carried out in the same way as in Reference Example 1, but using Adenovirus type 5 (ATCC VR-5). The results are shown in Table 3 and FIG. 3

TABLE 3
Results of the activity of the product LAE ®, batch 026297A,
against Adenovirus type 5 (ATCC VR-5), in clean conditions.

| | | | | | | | | Reduction with |
|---|---|---|---|---|---|---|---|---|
| | | | | log₁₀ TCID₅₀ after | | | | 95% confidence |
| | | Interfering | Level of | 0 | 15 | 30 | 60 | interval after |
| Product | Concentration | substance | cytotoxicity | min | min | min | min | 30 minutes |
| LAE ® | 1000 ppm | 0.3 g/L BSA | 0.5 | — | — | 0.50 | — | ≥5.91 ± 0.34 |
| | 500 ppm | | 0.5 | — | — | 1.66 | — | 4.75 ± 0.41 |
| | 50 ppm | | 0.5 | — | — | 4.49 | — | 1.92 ± 0.47 |
| Virus control | NA | 0.3 g/L BSA | NA | 6.50 | — | 6.41 | — | NA |
| Formaldehyde | 0.7% (m:v) | NA | 0.5 | NR | NR | 2.58 | 1.33 | NA |
| Formaldehyde | 0.7% (m:v) | NA | NA | 6.07 | NR | NR | 5.91 | NA |
| virus control | | | | | | | | |

Control of cell susceptibility (logarithmic difference between viral titers using treated and untreated cells) log10^{-0.42}
Control of the efficacy of suppression of product activity (logarithmic difference between virus titers of the control virus and that of the test suspension) log10^{-0.33}
NA: not applicable;
NR: not performed
Times recommended by standard for surfaces: maximum 5 or 60 min
Times recommended by standard for instruments: maximum 60 min
Times recommended by standard for hygienic friction hand treatment and hygienic hand washing: between 30 and 120 seconds
Viricidal activity exists when the virus titer shows a reduction ≥4 log₁₀.
PBS: phosphate saline buffer,
BSA: bovine serum albumin

Therefore, the disinfectant product LAE®, in clean conditions, at concentrations of 1000 ppm and 500 ppm and with 30 minutes exposure, has viricidal activity against Adenovirus type 5, with a reduction of ≥5.91±0.34 TCID₅₀ at the concentration of 1000 ppm, and with a reduction of 4.75±0.41 TCID₅₀ at the 500 ppm concentration.

However, LAE® has no viricidal activity at the concentration of 50 ppm against Adenovirus type 5, with a reduction of 1.92±0.47 TCID₅₀.

### Reference Example 3

### The Activity of LAE® was Investigated Against Murine Norovirus

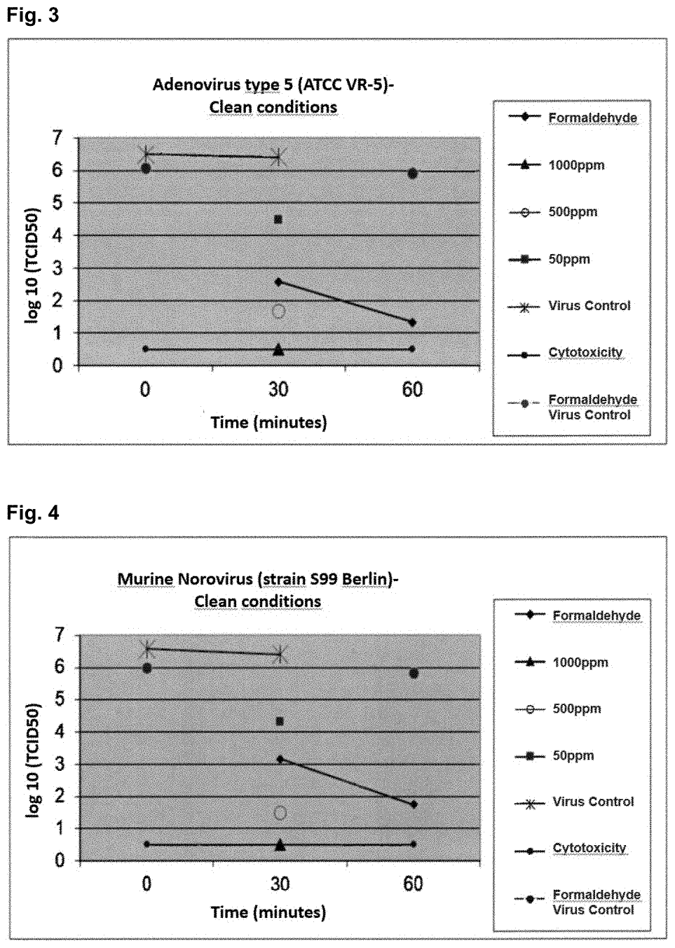

The test was carried out in the same way as in Example 1, but using murine Norovirus (cepa S99 Berlin). The contact time was 30 min. The incubation temperature was 37° C.±1° C. during 1 hour, the cellular line was Raw 264.7, Public Heath England, aliquots of work 7, passages 13 and 15, aliquots of work 8, passage 8. The results are shown in Table 4 and FIG. 4.

TABLE 4
Results of the activity of the product LAE ®, batch 026297A,
against murine Norovirus (cepa S99 Berlin), in clean conditions.

| | | | | | | | | Reduction with |
|---|---|---|---|---|---|---|---|---|
| | | | | log₁₀ TCID₅₀ after | | | | 95% confidence |
| | | Interfering | Level of | 0 | 15 | 30 | 60 | interval after |
| Product | Concentration | substance | cytotoxicity | min | min | min | min | 30 minutes |
| LAE ® | 1000 ppm | 0.3 g/L BSA | 0.5 | — | — | 0.50 | — | ≥5.91 ± 0.34 |
| | 500 ppm | | 0.5 | — | — | 1.49 | — | 4.92 ± 0.47 |
| | 50 ppm | | 0.5 | — | — | 4.32 | — | 2.09 ± 0.50 |
| Virus control | NA | 0.3 g/L BSA | NA | 6.58 | — | 6.41 | — | NA |
| Formaldehyde | 0.7% (m:v) | NA | 0.5 | NR | NR | 3.16 | 1.74 | NA |
| Formaldehyde | 0.7% (m:v) | NA | NA | 6.00 | NR | NR | 5.82 | NA |
| virus control | | | | | | | | |

Control of cell susceptibility (logarithmic difference between viral titers using treated and untreated cells) log10^{-0.58}
Control of the efficacy of suppression of product activity (logarithmic difference between virus titers of the control virus and that of the test suspension) log10^{-0.26}
NA: not applicable;
NR: not performed
Times recommended for surfaces: maximum 5 or 60 min
Times recommended for instruments: maximum 60 min
Times recommended for hygienic friction hand treatment and hygienic hand washing: between 30 and 120 seconds
Viricidal activity exists when the virus titer shows a reduction ≥4 log₁₀.
PBS: phosphate saline buffer,
BSA: bovine serum albumin

Thus, the disinfectant product LAE® in clean conditions, at concentrations of 1000 ppm and 500 ppm and with 30 minutes exposure, has viricidal activity against murine Norovirus, with a reduction of ≥5.91±0.34 TCID₅₀ at the concentration of 1000 ppm, and with a reduction of 4.92±0.47 TCID₅₀ at the 500 ppm concentration.

However, LAE® has no viricidal activity at the concentration of 50 ppm against murine Norovirus, with a reduction of 2.09±0.50 TCID₅₀.

1. A method of treatment of surfaces selected from the group consisting of the surface of human skin, the surface of food preparations, the surface of food contact materials, the surface of cosmetics, ground surface, the surface of any kind of vehicles, the surface of any equipment or textiles used in the handling of human beings or animals or human or animal skin, surfaces of hospital or medical practice equipment, hospital ground or medical practice ground surfaces, surfaces of public transport vehicles, surfaces of buildings or furniture, and surfaces of food areas, surfaces of medical devices such as medical tubing, stents, respiratory tubes, medical shields, visors, and medical protective gear to prevent or treat their contamination with Coronavirus,
characterized in that
a cationic surfactant of the following formula (1) 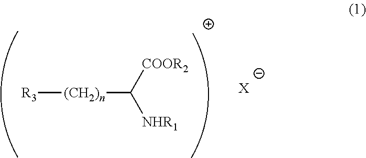
where:
X⁻ is Br⁻, Cl⁻, or HSO4, a counter ion derived from an organic or inorganic acids, or an anion on the basis of a phenolic compound;
R₁ is a linear alkyl chain from a saturated fatty acid or hydroxy acid from 8 to 14 atoms of carbon bonded to the α-amino acid group through an amidic bond,
R₂ is a linear or branched alkyl chain from 1 to 18 carbon atoms or an aromatic group,
R₃ is 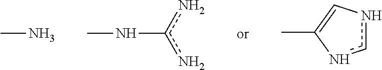
and n can be from 0 to 4,
is applied to the surface.

2. A method of treatment according to claim 1, wherein the compound of formula (1) is LAE® of formula (2), which is the ethyl ester of the lauramide of the arginine monohydrochloride:

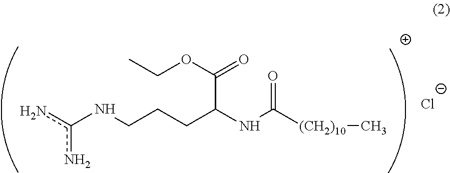

3. The method of claim 1 or 2, wherein the Coronavirus is Severe Acute Respiratory Syndrome Coronavirus 2 known as SARS-CoV-2 and/or Human Coronavirus 229E known as HCoV-229E.

4. The method of claims 1 to 3 for the treatment of constituents of food contact materials.

5. The method according to claim 4, wherein the constituents are polymeric plastics, wood, plywood, or paper or combinations thereof.

6. The method of claims 1 to 5, wherein the compound of formula (1) or (2) is applied as a formulation which is in the form of a solution, foam, spray, aerosol, cream, lotion, milk, emulsion, gel, hand-rub, skin treatment oil, beauty mask, bath salt, shampoo, hair conditioner, sanitizer gel, bath or shower oil, liquid soap, skin cleaning lotion, make-up or make-up remover.
